(19) United States
(12) Patent Application Publication
(10) Pub. No.: US 2021/0382051 A1
(43) Pub. Date: Dec. 9, 2021

(54) METHODS AND COMPOUND FOR THE IDENTIFICATION AND TREATMENT OF TUBERCULOSIS

(71) Applicants: Amanuel Tesfazion, Washington, DC (US); Lydia Seifu, Washington, DC (US); Meried Bezuneh, Alexandria, VA (US)

(72) Inventors: Amanuel Tesfazion, Washington, DC (US); Lydia Seifu, Washington, DC (US); Meried Bezuneh, Alexandria, VA (US)

(21) Appl. No.: 17/342,389

(22) Filed: Jun. 8, 2021

Related U.S. Application Data
(60) Provisional application No. 63/036,845, filed on Jun. 9, 2020.

(51) Int. Cl. G01N 33/569; A61P 31/06; A61K 36/18; A61K 31/235

(52) U.S. Cl. CPC G01N 33/5695; A61P 31/06; G01N 2333/35; A61K 31/235; A61K 36/18

(57) ABSTRACT

A method to diagnose bacterial infection, including phenotyping cell based resistant mycoplasm, amplification and identification of infection, and performing at least one assay on a cell culture.

### CROSS REFERENCE TO RELATED APPLICATION

This application claims the benefit of, and incorporates by reference, U.S. provisional patent application Ser. No. 63/036,845, entitled “Methods and Compound for the Identification and Treatment of Tuberculosis,” which was filed on Jun. 9, 2020.

### BACKGROUND

### 1. Field

The present general inventive concept relates generally to treatment of a bacterial disease, and particularly, to a methods and compound for the identification and treatment of tuberculosis.

### 2. Description of the Related Art

Tuberculosis (TB) is an ancient pandemic that has evolved from an ancestor between 40,000 to 70,000 years ago. TB has been affecting mankind for over 17,000 years and continues to be a global threat. The oldest known molecular evidence of TB was detected in a fossil of an extinct bison (Pleistocene bison), which was radiocarbon dated at 17,870±230 years, and also a 9000-year-old human remains which were recovered from a neolithic settlement in the Eastern Mediterranean. TB is found today in every country in the world, with the leading infectious cases of death worldwide.

The World Health Organization (WHO) estimates that 1.8 billion people, which is close to one quarter of the world's population are infected with Mycobacterium tuberculosis (M.tb), the bacteria that causes TB. Last year, 10 million fell ill from TB and 1.5 million died. TB is an airborne disease that can be spread by coughing or sneezing and is the leading cause of infectious disease worldwide. It is responsible for economic devastation and the cycle of poverty and illness that entraps families, communities, and even entire countries. Among the most vulnerable are women, children, and those with HIV/AIDS. There is growing resistance to available drugs, which means the disease is becoming more deadly and difficult to treat. There were more than half a million cases of drug resistant TB last year.

Symptoms of TB depend on where in the body the TB bacteria are growing. In the cases of pulmonary TB, it may cause symptoms, such as chronic cough, pain in the chest, hemoptysis (i.e. the coughing up of blood or blood-stained mucus from the bronchi, larynx, trachea, or lungs), weakness or fatigue, weight loss, fever, and night-sweats.

There are five Stages of tuberculosis:

(1) Onset (1-7 Days): The bacteria is inhaled.

(2) Symbiosis (7-21 Days): If the bacteria does not get killed then it reproduces.

(3) Initial Caseous Necrosis (14-21 Days): Tuberculosis starts to develop even when the bacteria slow down at reproducing because they kill the surrounding non-activated macrophages and run out of cells to divide in. The bacteria then produce anoxic (i.e. without oxygen) conditions and reduces the pH. The bacteria cannot reproduce anymore, but can live for a long time.

(4) Interplay of Tissue-Damaging and Macrophage Activating Immune Response (After 21 days): Macrophages will surround a Mycobacterium tuberculosis cell, but some may be inactive and/or a cell wall of the bacterium prevents fusion of the phagosome (i.e a vesicle formed around a particle engulfed by a phagocyte via phagocytosis) and lysosome (i.e. a membrane bound organelle that contain hydrolytic enzymes that can break down many kinds of biomolecules). Subsequently, the bacteria uses the macrophage to reproduce. The bacteria can break off and spread around. If it spreads in the blood you can develop tuberculosis outside the lungs, which is called miliary tuberculosis.

(5) Liquification and Cavity Formation: The bacterium at one point will liquify, which will make the disease spread faster, but most people will not get to this stage. Only a small percent of people will get to this stage.

FIG. 1 illustrates a life cycle development of Mycobacterium tuberculosis. (See http://www.histopathology-india.net/Tuberculosis.htm).

With the discovery of chemotherapy in the 1940s and adoption of the standardized short course in the 1980s, it was believed that TB would decline globally. Although, a declining trend was observed in most developed countries, this did not manifest to be true in many developing countries. It is the first infectious disease declared by the WHO as a global health emergency in 1993.

Ethiopia is one of the high TB burden countries in the world, with an estimated annual incidence and prevalence of 258 and 237 per 10⁵ population, respectively. The disease mainly affects people who are in the economically productive years of their life, which is usually between 15 and 59 years, thereby causing considerable social and economic burden on countries. Considering the aggravating factors, such as the human immunodeficiency virus (HIV and/or HIV-1) co-infection and the emerging drug resistance effective strategies for case detection and cutting transmission are urgently needed.

There are many countries which participate in a directly observed treatment, short course (DOTS) program. DOTS is the fastest expanding and the largest program in the world in terms of patients initiated on treatment, and the second largest, in terms of population coverage. Major challenges to control TB in many developing nations include poor primary health-care infrastructure in rural areas, unregulated private health care leading to widespread irrational use of first-line and second-line anti-TB drugs, spreading HIV infection, and in many cases a lack of political will.

Furthermore, multidrug-resistant TB (MDR-TB) is another emerging threat to TB eradication and is a result of deficient and/or deteriorating TB control programs. The WHO with its “STOP TB” strategy has given a vision to eliminate TB as a public health problem from the face of the Earth by 2050.

Despite newer modalities for diagnosis and treatment of TB, unfortunately, people are still suffering, and worldwide it is among the top ten killer infectious diseases, second only to HIV.

The emergence of MDR-TB has created a culture of Mycobacterium tuberculosis resistant to at least isoniazid (i.e. isonicotinic acid hydrazide or INH) and rifampicin (RMP), the two most powerful first-line treatment anti-TB drugs, has become a serious treatment problem. Several studies have shown that MDR-TB develops in otherwise treatable TB when the course of antibiotics is interrupted and the levels of drug in the body are insufficient to kill one-hundred percent of the bacteria. This can happen for a number of reasons: Patients may feel better and halt their antibiotic course, drug supplies may run out or become scarce, patients may forget to take their medication from time to time, and/or patients do not receive effective therapy.

Most tuberculosis therapy consists of short-course chemotherapy which is only curing a small percentage of patients with MDR-TB. Delays in second line drugs make MDR-TB more difficult to treat. MDR-TB is spread from person to person as readily as drug-sensitive TB and in the same manner. Even with the patient off second line anti-TB medication, the price is still high and therefore a big problem for patients living in poor countries to be treated. If patients were left untreated, the spread of tuberculosis would be problematic in poor countries. Moreover, complications from tuberculosis is often exacerbated with HIV co-infection. The two infectious vectors not only share the same residential target cell (e.g., macrophages), but also transactivate (i.e. activation of a gene at one locus by the presence of a gene at another locus, typically following infection by a virus) each other leading towards accelerated pathogenesis (i.e. manner of development of a disease). Conventional techniques such as acid, fast smear, and culture studies are either not sufficiently efficient and/or specific, or require an extended turnaround time from the laboratory.

Also, extra pulmonary tuberculosis is difficult to diagnose. As in many cases clinical, radiological, and laboratory findings are non-specific, and acid, fast smear and culture of acid-fast bacilli (AFB) are rarely positive (i.e. an AFB smear is a microscopic examination of a person's sputum or other specimen that is stained to detect acid-fast bacteria, such as Mycobacterium tuberculosis). Considering these factors, such as drug resistance, TB/HIV co-infection and the scale of the epidemics, an effective diagnostic system and appropriate drug intervention is urgently needed.

Therefore, there is an immediate need for an effective remedy that is natural, inexpensive, and non-toxic. As such, there is a need for methods and compound for the identification and treatment of tuberculosis.

### SUMMARY

The present general inventive concept provides methods and compound for the identification and treatment of tuberculosis.

Additional features and utilities of the present general inventive concept will be set forth in part in the description which follows and, in part, will be obvious from the description, or may be learned by practice of the general inventive concept.

The foregoing and/or other features and utilities of the present general inventive concept may be achieved by providing a method to diagnose bacterial infection, including phenotyping cell based resistant mycoplasm, amplification and identification of infection, and performing at least one assay on a cell culture.

The amplification may be a shell vial spin amplification method.

The identification may be a direct infected co-culture method, such that co-infection includes HIV.

The at least one assay may be based on ELISA.

The at least one assay may include at least one of ELISA IgG, IgM, and IgA.

The at least one assay may be based on ERBA LISA and another at least one assay is SEVA TB ELISA, which are performed simultaneously.

The bacterial infection may be caused by Mycobacterium tuberculosis.

The foregoing and/or other features and utilities of the present general inventive concept may also be achieved by providing a method for the treatment of a bacterial disease, including administering to a subject in need thereof of an anti-pathogenic compound, such that the anti-pathogenic compound is derived from an herbal extract.

The herbal extract may be a glycol derivative.

The glycol derivative may be diethylene glycol dibenozate.

The bacterial disease may be caused by Mycobacterium tuberculosis.

The anti-pathogenic compound may boost an immune system of the subject.

The anti-pathogenic compound may boost the immune system by stimulating production of gamma interferon.

The anti-pathogenic compound may boost the immune system by inhibiting a protease enzyme of the bacterial disease.

The anti-pathogenic compound may boost the immune system by upregulating cellular genes.

### BRIEF DESCRIPTION OF THE DRAWINGS

These and/or other features and utilities of the present generally inventive concept will become apparent and more readily appreciated from the following description of the embodiments, taken in conjunction with the accompanying drawings of which:

FIG. 1 illustrates a life cycle development of Mycobacterium tuberculosis;

FIG. 2A illustrates a molecular structure of an anti-pathogenic compound, known as 90I (diethylene glycol dibenzoate), according to an exemplary embodiment of the present general inventive concept;

FIG. 2B illustrates a molecular structure of the anti-pathogenic compound, known as 90I, according to an exemplary embodiment of the present general inventive concept;

FIG. 3 illustrates a graph showing production of gamma interferon by cells receiving 90I compared to other drugs, according to an exemplary embodiment of the present general inventive concept;

FIG. 4A illustrates a surface view of 90I interacting with residues GLY 110 and THR 125, according to an exemplary embodiment of the general present inventive concept;

FIG. 4B illustrates the surface view of a first pose of 90I in white with an H bond interaction and an active site in blue, according to an exemplary embodiment of the general present inventive concept;

FIG. 4C illustrates the surface view of 90I in white disposed deep inside the active site, according to an exemplary embodiment of the general present inventive concept;

FIG. 4D illustrates the surface view of 90I in white disposed deep inside the active site with three H bonds, according to an exemplary embodiment of the general present inventive concept;

FIG. 5A illustrates 90I in white disposed within a binding pocket interfering with Mycobacterium tuberculosis by interacting with active site residues, according to an exemplary embodiment of the general present inventive concept;

FIG. 5B illustrates 90I in white disposed within the binding pocket and interacting with a plurality of binding site residues in blue, according to an exemplary embodiment of the general present inventive concept;

FIG. 6 illustrates 90I in white interacting with residues THR 491 and HIS 490, in blue, including two H bonds, according to an exemplary embodiment of the general present inventive concept;

FIG. 7A illustrates 90I in white interacting with a residue THR 125 in blue of the active site, according to an exemplary embodiment of the general present inventive concept;

FIG. 7B illustrates a surface view of 90I in white disposed in a binding pocket interacting with the residue THR 125 in blue of the active site, according to an exemplary embodiment of the general present inventive concept;

FIG. 8 illustrates another pose of 90I interacting with a residue GLN 495 in blue of the active site including two H bonds, according to an exemplary embodiment of the general present inventive concept;

FIG. 9 illustrates 90I interacting with a plurality of residues including SER 228, TYR 342, and HIS 490 with two H bonds, according to an exemplary embodiment of the general present inventive concept;

FIG. 10 illustrates 90I interacting with a plurality of residues GLY 110, SER 228, and THR 491 connected to HIS 490 including three H bonds, according to an exemplary embodiment of the general present inventive concept;

FIG. 11 illustrates 90I interacting with a plurality of residues GLN 495 and TYR 227 connected to SER 228, according to an exemplary embodiment of the general present inventive concept;

FIG. 12 illustrates 90I disposed within a binding pocket interacting with a residue GLN 495, according to an exemplary embodiment of the general present inventive concept;

FIG. 13 illustrates 90I penetrating a binding pocket, according to an exemplary embodiment of the general present inventive concept;

FIG. 14 illustrates 90I disposed within a binding pocket interfering with an active site residue GLN 495, according to an exemplary embodiment of the general present inventive concept;

FIG. 15 illustrates 90I disposed deep within a binding pocket interacting with a plurality of active site residues, according to an exemplary embodiment of the general present inventive concept;

FIG. 16 illustrates a different pose of 90I disposed within a binding pocket, according to an exemplary embodiment of the general present inventive concept; and

FIG. 17 illustrates 90I disposed within a binding pocket, according to an exemplary embodiment of the general present inventive concept.

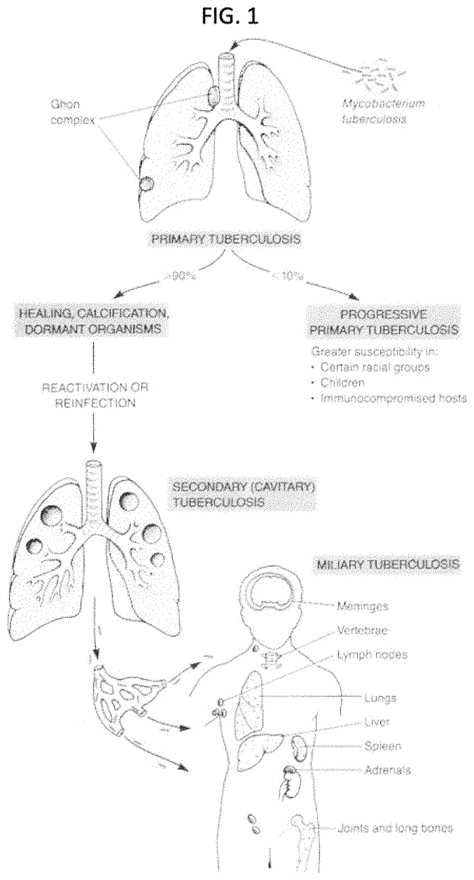

### DETAILED DESCRIPTION

Various example embodiments (a.k.a., exemplary embodiments) will now be described more fully with reference to the accompanying drawings in which some example embodiments are illustrated. In the figures, the thicknesses of lines, layers and/or regions may be exaggerated for clarity.

Accordingly, while example embodiments are capable of various modifications and alternative forms, embodiments thereof are shown by way of example in the figures and will herein be described in detail. It should be understood, however, that there is no intent to limit example embodiments to the particular forms disclosed, but on the contrary, example embodiments are to cover all modifications, equivalents, and alternatives falling within the scope of the disclosure. Like numbers refer to like/similar elements throughout the detailed description.

It is understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present. Other words used to describe the relationship between elements should be interpreted in a like fashion (e.g., “between” versus “directly between,” “adjacent” versus “directly adjacent,” etc.).

The terminology used herein is for the purpose of describing particular embodiments only and is not intended to be limiting of example embodiments. As used herein, the singular forms “a,” “an” and “the” are intended to include the plural forms as well, unless the context clearly indicates otherwise. It will be further understood that the terms “comprises,” “comprising,” “includes” and/or “including,” when used herein, specify the presence of stated features, integers, steps, operations, elements and/or components, but do not preclude the presence or addition of one or more other features, integers, steps, operations, elements, components and/or groups thereof.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art. However, should the present disclosure give a specific meaning to a term deviating from a meaning commonly understood by one of ordinary skill, this meaning is to be taken into account in the specific context this definition is given herein.

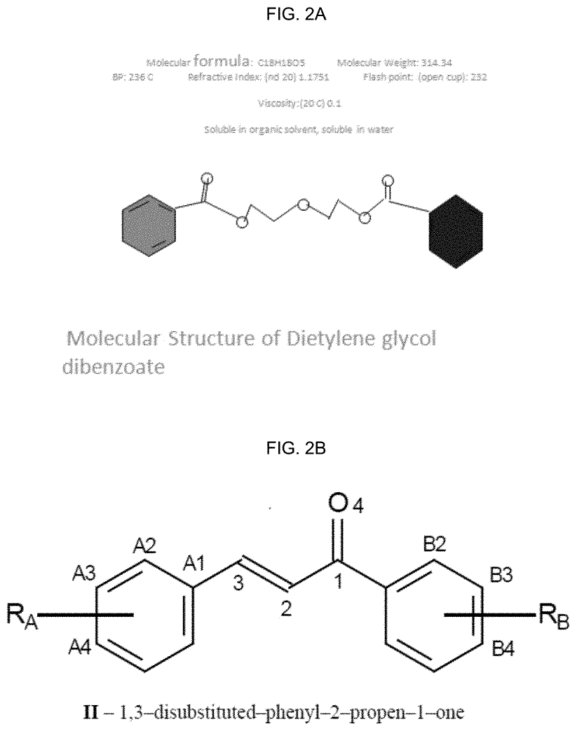

FIG. 2A illustrates a molecular structure of an anti-pathogenic compound, known as 90I (diethylene glycol dibenzoate), according to an exemplary embodiment of the present general inventive concept.

FIG. 2B illustrates a molecular structure of the anti-pathogenic compound, known as 90I, according to an exemplary embodiment of the present general inventive concept.

Referring to FIGS. 2A and 2B, an anti-pathogenic agent and/or the anti-pathogenic compound may be identified as 90I (or 90i). The anti-pathogenic compound may be derived from the herbal extract identified as H2K1001. H2K1001 and/or 90I may be a natural product that was isolated using the Bioassay Guided Fractionation, and further purified, molecularly characterized (i.e. characterizing at the molecular level without any effect of environment or development or physiological state of the organism), and not only found to be highly potent against all HIV strains, but also immunogenic with unique multiple modes of action (i.e. functional or anatomical change at a cellular level, resulting from exposure of a living organism to a substance), potently effective against both reverse transcriptase and a protease (PR) enzyme (i.e. an enzyme which breaks down proteins and peptides). The essence of combination drug therapy, HAART regiments, may be its effectiveness against all HIV-1 strains that is potent enough to bring the viral load down to an undetectable level. This may be achieved by combining an RT and a PR combination synergy to affect multiple modes of action.

Furthermore, although the pathogen is identified as Mycobacterium tuberculosis, 90I may be used to treat any pathogen including a virus, bacteria, protozoan (i.e. parasite), and/or fungal.

Bacterial pathogens may include Mycobacterium tuberculosis Tuberculosis, Bacillus anthracis Anthrax, and Staphylococcus Sepsis aureus, but is not limited thereto.

Viral pathogens may include Adenoviridae, Mastadenovirus, Infectious canine hepatitis, Arenaviridae, Arenavirus, Lymphocytic choriomeningitis, Caliciviridae, Norovirus, Norwalk virus infection, Coronaviridae, Coronavirus, Severe Acute Respiratory Syndrome, SARS-CoV, SARS-CoV-2, Torovirus, Filoviridae, Marburgvirus, Viral hemorrhagic fevers, Ebolavirus, Viral hemorrhagic fevers, Flaviviridae, Flavivirus, West Nile Encephalitis, Hepacivirus, Hepatitis C virus infection, Pestivirus, Bovine Virus Diarrhea, Classical swine fever, Hepadnaviridae, Orthohepadnavirus, Hepatitis, Herpesviridae, Simplexvirus, cold sores, genital herpes, bovine mammillitis, Varicellovirus, chickenpox, shingles, abortion in horses, encephalitis in cattle, Cytomegalovirus, infectious mononucleosis, Mardivirus, Marek's disease, Orthomyxoviridae, Influenzavirus A, Influenza, Influenzavirus B, Influenza, Papillomaviridae, Papillomavirus, Skin warts, skin cancer, cervical cancer, Picornaviridae, Enterovirus, Polio, Rhinovirus, Common cold; Aphthovirus, Foot-and-mouth disease, Hepatovirus, Hepatitis, Poxviridae, Orthopoxvirus, Cowpox, vaccinia, smallpox, Reoviridae, Rotaviruses, Diarrhea, Orbivirus, Blue tongue disease, Retroviridae Gammaretrovirus, Feline leukemia, Deltaretrovirus, Bovine leukemia, Lentivirus, Human immunodeficiency, FIV, and SIV, Rhabdoviridae, Lyssavirus, Rabies, Ephemerovirus, Bovine ephemeral fever, Togaviridae, Alphavirus, and Eastern and Western equine encephalitis, but is not limited thereto.

Parasitic pathogens may include Plasmodium, Malaria, Leishmania, and Leishmaniasis, but is not limited thereto.

Fungal pathogens may include Aspergillis, Candida, Coccidia, Cryptococci, Geotricha, Histoplasma, Microsporidia, and Pneumocystis, but is not limited thereto.

As such, 90I may also be an anti-pathogenic compound that is applicable to different diseases and/or infections.

The Ethiopian region may be characterized by a wide range of ecological, edaphic, and climatic conditions that account for the wide diversity of its biological resources, both in terms of flora and faunal wealth. The plant genetic resources of the country exhibit an enormous diversity as seen in the fact that Ethiopia is one of the twelve Vavilov Centers of origin for domesticated crops and their wild and weedy relatives. According to recent studies, it is estimated that there are more than seven thousand species of flowering plants recorded in Ethiopia, of which at least twelve percent are probably endemic.

Medicinal plants may comprise one of the important components of Ethiopian vegetation. On record, there may be six hundred species of medicinal plants constituting a little over ten percent of Ethiopia's vascular flora. The medicinal plants may be distributed all over the country, with greater concentration in the south and southwestern parts of the country. Woodlands of Ethiopia may be the source of most of the medicinal plants, followed by the montane grassland and/or dry montane forest complex of the plateau. Other important vegetation types for medicinal plants may be the evergreen bushland and rocky areas.

As such, an herbal extract may be extracted from the herb from Ethiopia. The herbal extract may include a glycol derivative. Moreover, the glycol derivative may include diethylene glycol dibenzoate. An anti-pathogenic compound may include diethylene glycol dibenzoate to treat tuberculosis.

There are several objectives to be developed during development of treatment including a cost effective diagnostic system involving cell based resistant mycoplasm phenotyping, infection for amplification and identification, a home made enzyme-linked immunosorbent assay (ELISA) system (i.e. a plate based assay technique designed for detecting and quantifying soluble substances such as peptides, proteins, antibodies, and hormones, a validation assay that will include other standard assays, ELISA IgG (i.e. a widely expressed serum antibody, ELISA will measure a target protein in biological samples), IgM (i.e. immunoglobulin M is one of several types of antibody that are produced by vertebrates), IgA Assay (i.e. assay that measures the amount of target bound between a matched antibody pair). ERBA LISA (TB IgG) Test (i.e. a in-vitro diagnostic kid for qualitative determination of total antibodies, IgG) and SEVA TB (IgG) ELISA Test Shifts (i.e. multi-antigen and antibody assay) in susceptibility for clinical isolate are measured by determining the EC50 values for the isolate and WT standard mycobacterium done under the same condition and at the same time. Simultaneous testing provides for absolute comparisons between assays.

Clinical diagnostics of TB in a country like Ethiopia should considered: a) problems of resistance to the current commercial drugs, b) problems of co-infection with HIV-1, and/or c) cost effectiveness. The clinical diagnostic system proposed in this research study not only measures up to the above criteria, but also quantitates mycobacterium and HIV-1 directly from the target cells, macrophages. This study includes (1) Direct Isolation, Quantitation, Resistant Surveillance of Mycobacterium (DIQRSM) from a co-infected macrophage (e.g., with HIV), and (2) an additional new tissue culture system, shell vial spin amplification.

Purpose of the Direct Infected Macrophage Co-Culture Method

This procedure describes the general method to be used to isolate, expand, and conduct drug resistant surveillance of infectious mycobacterium from clinical specimens by co-culture method from primary macrophage cells. The principle in this procedure involves isolating infected macrophage directly from patients by extracting 10 milliliters (ml) of whole blood using density gradient ficoll fractionation (i.e. separation and concentration of parasitized erythrocytes from infected blood by centrifugation of a sample) and seeding it on to NHS primed flat bottom plate with uninfected monocyte target cells from ser-negative blood for co-culture multiplication of mycobacterium and HIV. Post amplification of the dual micro-organisms and titered with determination of median tissue culture infectious dose (TCID50) (i.e. concentration at which 50% of cells are infected when a test tube or well plate upon which cells have been cultured is inoculated with a diluted solution of viral fluid), drug phenotyping and/or resistant surveillance test could easily be determined. Therefore, the procedure will provide a summary of Direct Isolation, Quantitation, Resistant Surveillance of Mycobacterium (DIQRSM) from co-infected macrophage.

Purpose of Shell Vial Spin Amplification Method

Rapid detection of Mycobacterium from a clinical specimen is essential for timely therapeutic intervention against Mycobacterium tuberculosis, as well as, reversing AIDS associated pulmonary complications.

HIV-1 infection of the lung involves alveolar macrophages which also get co-infected by mycobacterium during HIV-1 pathogenesis. The co-infection may include: a) transactivation of HIV-1 replication by a thousand fold, depleting the patients CD4 on one hand and b) creating a fertile environment for mycobacterium to replicate in the same target cell, alveolar macrophages, together causing pulmonary complications of AIDS patients. In other words, HIV-1 infection facilitates infection by Mycobacterium tuberculosis due to weakened macrophages.

A conventional system of culturing sputum of a patient not only has a long incubation period (e.g., close to two months) to detect, but it is also inconvenient to handle many specimens at the same time.

Shell vial-spin amplified cell culture assay system offers several advantages over the conventional system for at least the following reasons: a) the assay is highly sensitive because mycobacterium grows better and faster in its natural target cell, as well as, the spin force facilitates adhesion by every micro-organism therein, such that infection and entry in to the cell membrane may occur very easily, and b) the turnaround time for detection of positive culture is significantly reduced to five days.

90I has been observed to provide multiple modes of action. More specifically, 90I may inhibit a protease enzyme, increase production of gamma interferon, and/or improve upregulation (i.e. the process of increasing a response to a stimulus, such as a cellular response to a molecular stimulus due to increase in the number of receptors on a cell surface) cellular genes translates to a strong anti-malaria and anti-TB novel drugs, respectively and simultaneously.

As discussed previously, MDR-TB is resistant to at least isoniazid and rifampicin. As such, an alternative drug that may be effective against sensitive and MDR-TB is in high demand. The Therapeutic Index (TI) value of 90I against TB surpasses currently available treatments. It has been well studied that pretreatment of macrophages with recombinant gamma interferon (IFN) prevents HIV-1 and Mycobateriums lipopolysaccharide (LPS) replication acting at late stage in the viral infection cycle (See Kornbluth et al., 1989). The downregulation (i.e. the process of reducing or suppressing a response to a stimulus, such that cellular response to a molecule is due to a decrease in the number of receptors on a cell surface) of gamma interferon benefits the virus and/or bacteria (e.g., HIV, Mycobacterium tuberculosis) because the macrophage effector functions (i.e. a major component of an anti-pathogen defense system and/or an immune system response by macrophages may include phagocytosis and/or cytokine production) is compromised, both directly by the TH2 cytokines IL4 and IL10 and indirectly by suppressions of gamma interferon secretions by TH1 cells (See Sher et al., 1992).

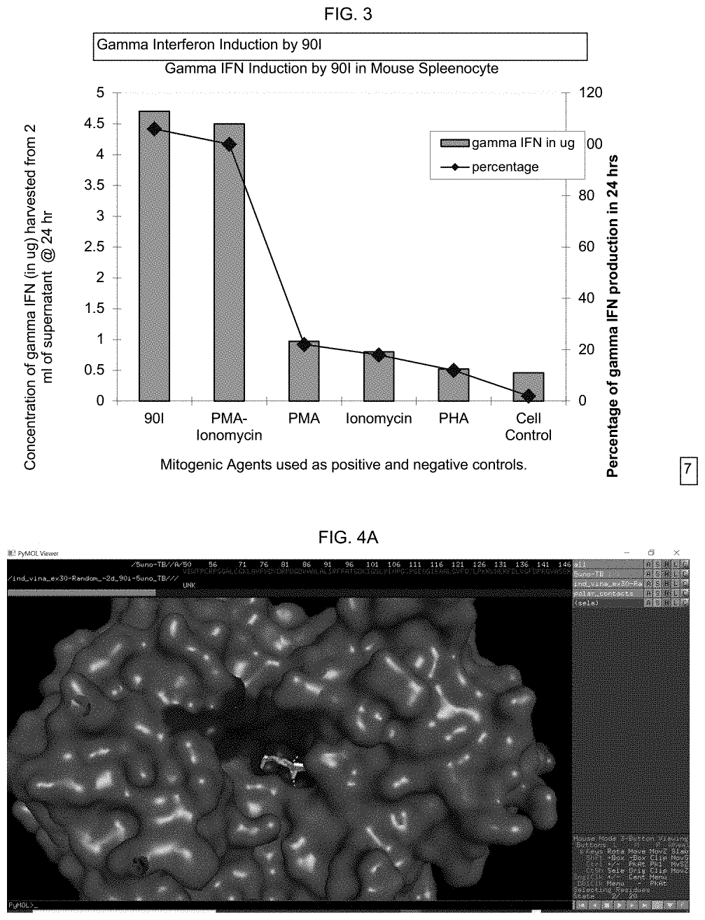

FIG. 3 illustrates a graph showing production of gamma interferon by cells receiving 90I compared to other drugs, according to an exemplary embodiment of the present general inventive concept.

Referring to FIG. 3, this assay may underscore 90I's absorptions in a monocyte and/or a macrophage primary cell, such that the anti-pathogenic compound may have stability and longer pharmacokinetic half-life in ten days assay with single time point drug addition. Another significance of this result is that 90I and/or HK1001 may reinstate a dysfunctional monocyte to resume its natural functional role as a primary effector cell in the cellular immune system, effecting extensive anti-microbial and/or anti-fungal functional capability in the killing of multiple pathogens and/or other opportunistic infecting agents, such as Mycobacterium tuberculosis.

Moreover, activated CD8+ cells are reported to produce high levels of gamma interferon, which may be involved in the anti-TB immune responses, contributing to both control of bacterial spread and concomitant lymphoid follicular lyses. An amount of gamma interferon produced by 90I may be equivalent to that of the positive control, PMA-lonomycin combination. The conclusion from this result may be that 90I stimulates cellular genes to produce gamma interferon. This finding may have a far-reaching implication and relevant in that H2K1001 and/or 90I has the potential in the restoration of immune competence, a strong immune modulator. 90I may be as strong as a vaccine because it may modulate the immune cell signal switch from Th2 to Th1 (i.e. a subset of T lymphocytes that express CD4 and are known as T-helper cells, they produce cytokines, specifically Th1-type cytokines). T-helper subset population Th1 and Th2 subset have been identified in animals and humans based on cytokines secreted. Th1 subsets favors cellular immune response by secreting cellular factors, such as IL-2, gamma interferon and interleukin 12 (IL-12) (i.e. a cytokine that is produced by activated antigen-presenting cells, such as dendritic cells and/or macrophages). The Th2 subset may favor a humoral response, including IL-4, IL-5, and IL-6 and causes activation of B cells (i.e. B lymphocytes) leading to antibody formations.

Furthermore, Th1 provides a strong immunological response. This study shows that 90I is not only a potent antiviral, but also an immune system booster.

Research Experimental Design for 90I Evaluation Against TB

Materials

Mycobacterium Resistant Strains: Multi-drug-resistant tuberculosis (MDR-TB), resistant to at least isonizid (INH) and rifampicin (RMP) from ATCC

Test Drugs: a) 90I b) Isonized (INH) c) Rifampcin (RMP) from Pharmaceuticals.

Primary Cell: Monocyte will be isolated from HIV-1 and TB negative blood donors.

End point Determination: IFN.

Media (RPMI, FBS, L. glutamin).

Preparation to Testing

Enough Monocyte will be harvested and cryo preserved at cell density of 10×106/vial.

Mycobacterium will be isolated, Ethiopian patients expanded and tittered on Monocyte.

Resistant Mycobacterium will be expanded in the presence of their respective drug.

Drugs including 90I, Isoniazid (INH) and Rifampicin will be prepared @4000× stored at −70 C.

Functionality of the Test System

Each Test System Should Include Proper Control:

Drug dilution steps+Mycobacterium+Cell in triplicate wells for each drug dilutions, Drug Efficacy.

Drug dilution steps+Cell in triplicate wells for each drug dilutions, Toxicity Control.

Mycobacterium only Positive Control.

Cell only Negative Control.

Experimental Setup on Flat Bottom 96 Well Bottom

Test Drug I: 90I Vs Mycobacterium (Sensitive)

TABLE 1
Drug Evaluation Study Design

| A | 1 | 2 | 3 | 4 | 5 | 6 | 7 | 8 | 9 | 10 |
|---|---|---|---|---|---|---|---|---|---|---|
| B | | | | | | | | | | |
| C | Test Drug (90I) + Infectious Micro-Organism + Cells in triplicate wells | | | | | | | | | |
| D | | | | | | | | | | |
| E | | | | | | | | | | |
| F | Test Drug + Cells only Toxicity Control for Test Drug in triplicate wells | | | | | | | | | |
| G | | | | | | | | | | |
| H | Saline buffer or just Media used as heat vaporization control | | | | | | | | | |
| | 3.18E−07M | 1.06E-07M | 3.54E−08M | 1.18E−08M | 3.93E−09M | 1.31E−09M | 4.37E−10M | 1.46E−10M | 4.85−11M | |

Drug dilutions in half log
Drug concentration in Molarity

Referring to Table 1, data intended for measurement during testing of 90I against sensitive TB.

Test Drug II: 90I Vs Mycobacterium (Resistant-MDR-TB)

TABLE 2
Drug Evaluation Study Design

| A | 1 | 2 | 3 | 4 | 5 | 6 | 7 | 8 | 9 | 10 |
|---|---|---|---|---|---|---|---|---|---|---|
| B | | | | | | | | | | Pos Con |
| C | Test Drug (90I) + Infectious Micro-Organism + Cells in triplicate wells | | | | | | | | | Pos Con |
| D | | | | | | | | | | Pos Con |
| E | | | | | | | | | | Cell Con |
| F | Toxicity Control: Test Drug + Cells only in triplicates | | | | | | | | | Cell Con |
| G | | | | | | | | | | Cell Con |
| H | Saline buffer or just Media used as heat vaporization control | | | | | | | | | |
| | 3.18E−07M | 1.06E-07M | 3.54E−08M | 1.18E−08M | 3.93E−09M | 1.31E−09M | 4.37E−10M | 1.46E−10M | 4.85−11M | |

Drug dilutions in half log
Drug concentration in Molarity

Referring to Table 2, data intended for measurement during testing of 90I against MDR TB.

Test Drug 3: Isoniazid Vs Mycobacterium (Sensitive)

TABLE 3
Drug Evaluation Study Design

| A | 1 | 2 | 3 | 4 | 5 | 6 | 7 | 8 | 9 | 10 |
|---|---|---|---|---|---|---|---|---|---|---|
| B | | | | | | | | | | |
| C | Test Drug (90I) + Infectious Micro-Organism + Cells in triplicate wells | | | | | | | | | |
| D | | | | | | | | | | |
| E | | | | | | | | | | |
| F | Test Drug + Cells only Toxicity Control for Test Drug in triplicate wells | | | | | | | | | |
| G | | | | | | | | | | |
| H | Saline buffer or just Media used as heat vaporization control | | | | | | | | | |
| | 3.18E−07M | 1.06E-07M | 3.54E−08M | 1.18E−08M | 3.93E−09M | 1.31E−09M | 4.37E−10M | 1.46E−10M | 4.85−11M | |

Drug dilutions in half log
Drug concentration in Molarity

Referring to Table 3, data intended for measurement during testing of isoniazid against sensitive TB.

Test Drug 4 Isoniazid Vs Mycobacterium (Resistant MDR-TB)

TABLE 4
Drug Evaluation Study Design

| A | 1 | 2 | 3 | 4 | 5 | 6 | 7 | 8 | 9 | 10 |
|---|---|---|---|---|---|---|---|---|---|---|
| B | | | | | | | | | | Pos Con |
| C | Test Drug (Isonized) + Infectious Micro-Organism + Cells in triplicate wells | | | | | | | | | Pos Con |
| D | | | | | | | | | | Pos Con |
| E | Test Drug + Cells only Toxicity Control for Test Drug in triplicate wells | | | | | | | | | Cell Cont |
| F | | | | | | | | | | Cell Cont |
| G | | | | | | | | | | Cell Cont |
| H | Saline buffer or just Media used as heat vaporization control | | | | | | | | | |
| | 3.18E−07M | 1.06E-07M | 3.54E−08M | 1.18E−08M | 3.93E−09M | 1.31E−09M | 4.37E−10M | 1.46E−10M | 4.85−11M | |

Drug dilutions in half log
Drug concentration in Molarity

Referring to Table 4, data intended for measurement during testing of isoniazid against MDR-TB.

Test Drug 5: Rifampicin Vs Mycobacterium (Sensitive)

TABLE 5
Drug Evaluation Study Design

| A | 1 | 2 | 3 | 4 | 5 | 6 | 7 | 8 | 9 | 10 |
|---|---|---|---|---|---|---|---|---|---|---|
| B | | | | | | | | | | Pos Con |
| C | Test Drug (Rifampicin) + Infectious Micro-Organism + Cells in triplicate wells | | | | | | | | | Pos Con |
| D | | | | | | | | | | Pos Con |
| E | | | | | | | | | | Cell Cont |
| F | Test Drug + Cells only Toxicity Control for Test Drug in triplicate wells | | | | | | | | | Cell Cont |
| G | | | | | | | | | | Cell Cont |
| H | Saline buffer or just Media used as heat vaporization control | | | | | | | | | |
| | 3.18E−07M | 1.06E-07M | 3.54E−08M | 1.18E−08M | 3.93E−09M | 1.31E−09M | 4.37E−10M | 1.46E−10M | 4.85−11M | |

Drug dilutions in half log.
Drug concentration in Molarity

Referring to Table 5, data intended for measurement during testing of rifampicin against sensitive TB.

Test Drug 6 Rifampicin Vs Mycobacterium (Resistant MDR-TB)

TABLE 6
Control drug I Evaluation against Mytobacterium resistant

| A | 1 | 2 | 3 | 4 | 5 | 6 | 7 | 8 | 9 | 10 |
|---|---|---|---|---|---|---|---|---|---|---|
| B | | | | | | | | | | Pos |
| C | Test Drug (Rifampicin) + Infectious Micro-Organism + Cells in triplicate | | | | | | | | | Cont. |
| D | | | | | | | | | | |
| E | | | | | | | | | | Cell |
| F | Test Drug + Cells only Toxicity Control for Test Drug in triplicate wells | | | | | | | | | Cont |
| G | | | | | | | | | | |
| H | Saline buffer or just Media used as heat vaporization control | | | | | | | | | |
| | 3.18E−07M | 1.06E-07M | 3.54E−08M | 1.18E−08M | 3.93E−09M | 1.31E−09M | 4.37E−10M | 1.46E−10M | 4.85−11M | |

Drug dilutions in half log
Drug concentration in Molarity

Referring to Table 6, data intended for measurement during testing of rifampicin against MDR-TB.

The following is an excerpt from “Detection of Anti-Interferon-Gamma Autoantibodies in Subjects Infected by Mycobacterium Tuberculosis.” (See https://pubmed.ncbi.nlm.nih.gov/9562113/).

Setting: Among the cytokines involved in defensive mechanisms against Mycobacterium tuberculosis infection, special attention has been given to interferon-gamma (IFN-gamma); a local synthesis of this cytokine as well as IL-2 (type 1 cytokines) at the site of disease in patients with tuberculous pleuritis has been demonstrated. Moreover, high levels of IgG autoantibodies against IFN-gamma have been shown in several clinical situations. It has been suggested that these antibodies could serve to limit the intensity or duration of the immune response or be able to interfere with the pathophysiological effects of IFN-gamma.

Objective:

To investigate the potential role of anti-IFN-gamma antibodies in the course of M. tuberculosis infection.

Design:

Investigation of the presence of these antibodies in sera from healthy and ill subjects infected with M. tuberculosis in relation to the extent of the disease and the presence of IFN-gamma in sera by enzyme-linked-immunosorbent assay (ELISA). In order to investigate the presence of these antibodies at the site of infection we included 12 pleural fluids from tuberculosis patients and 9 pleural fluids from other origins.

Results:

In the course of M. tuberculosis infection the production of anti-IFN-gamma IgG antibodies is induced, being particularly higher in healthy skin test converters. Among tuberculosis patients, the presence of anti-IFN-gamma autoantibodies is significantly associated with detectable levels of the cytokine in sera. Levels of anti-IFN-gamma antibodies in moderately advanced and far advanced tuberculosis patients are significantly greater than in healthy individuals. These antibodies increase at the site of infection.

### Conclusion

Anti-IFN-gamma antibodies must be considered as a new element in the immune response to M. tuberculosis. It would be of great interest to investigate this point especially at the site of infection.

Tabular, Statistical Data Analysis and Dose Response Curves

TABLE 7
CONTROL SUMMARY

| | | | | | | | | | | |
|---|---|---|---|---|---|---|---|---|---|---|
| Drug Co | Mean | StDev. | CV | IFN Gama | IFN Gama | Drug Conc. | Mean | StDev. | CV | % CC |

Test Summary

| Infected Cells | | | | | Uninfected Cells | | | | |
|---|---|---|---|---|---|---|---|---|---|
| Drug Co | Mean | StDev. | CV | % p24R | Drug Conc. | Mean od | StDev. | CV | % CC |
| 0 | | | | | | | | | |
| [symbol] | | | | | | | | | |
| [symbol] | | | | | | | | | |
| [symbol] | | | | | | | | | |
| [symbol] | | | | | | | | | |
| [symbol] | | | | | | | | | |
| [symbol] | | | | | | | | | |
| [symbol] | | | | | | | | | |
| [symbol] | | | | | | | | | |
| [symbol] | | | | | | | | | |

Statistical Abbreviations and Explanations

| | | | |
|---|---|---|---|
| EC50: | Effective Cocentration 50%; Concentration | Mean: | Arithemetic Average of p24 or MTS |
| | Causing 50% inhibition of the virus | | reduction value colormetric Data, 3 rep. |
| IC50: | inhibitory Concentration 50% Concentration | StDev: | Sample standard deviation of Mean; 3 rep |
| | Causing 50% inhibition of the virus | TLD50: | 50% Tissue Culture Lethal Dosage |
| TI50: | Therapeutic Index; IC50/EC50 | CV: | Coeficient of Varience of the Mean; |
| % p24R: | % inhibition; 100 (100 * Mean)/Virus control | | StDev/Mean |
| MOI: | Multiplicity of Infection | % CC: | % cell control; 100 * Mean/Cell ctl |
| HT: | Drugs High Test Concentration | DF: | Drug dilution factor |
| DS: | Dilution Step between high test and low test | MDR: | Multi drug resistant TB Strains |
| SI: | Syncytium inducer | NSI: | Non Syncytium inducer |
| CLI: | Clinical Isolate | SL: | Slow permisive viral kinetic replication |
| Rapid [symbol] | Fast Viral kinetic replication | Monocyte: | Primary Cell Isolated from Human |

[symbol]

indicates data missing or illegible when filed

Referring to Table 7, data intended for measurement during testing of 90I.

The fundamental underlying advantage that 90I has in comparison to the current treatments used for hepatitis C may include a flavonoid phytochemical effective anti-oxidant that may prevent liver cancer, multiple molecular modes of action that parallels to not one, but all currently used treatments (i.e. protease inhibitors and interferon producer), multiple natural lead isolates identified, multiple modes of application, highly active (HAART), proven effective against resistance, such that promoting use of this drug without the need of combinatorial drugs being required, a natural product, provides a boost to the immune system, reverse latent infection, highly effective in brain cells, non-toxic, and affordable, but is not limited thereto.

In-Silico Analysis of 90I Against Mycobacterium tuberculosis

90I may inhibit Mycobacterium tuberculosis serine protease by interacting and interfering with the substrate binding site residues and should act as a ligand that is an inhibitor.

The following is an excerpt from “In silico analyses for the discovery of tuberculosis drug targets.” (See https://doi.org/10.1093/jac/dkt273).

Antibacterial drug discovery is moving from largely unproductive high-throughput screening of isolated targets in the past decade to revisiting old, clinically validated targets and drugs, and to classical black-box whole-cell screens. At the same time, due to the application of existing methods and the emergence of new high-throughput biology methods, we observe the generation of unprecedented qualities and quantities of genomic and other omics data on bacteria and their physiology. Tuberculosis (TB) drug discovery and biology follow the same pattern. There is a clear need to reconnect antibacterial drug discovery with modern, genome-based biology to enable the identification of new targets with high confidence for the rational discovery of new drugs. To exploit the increasing amount of bacterial biology information, a variety of in silico methods have been developed and applied to large-scale biological models to identify candidate antibacterial targets. Here, we review key concepts in network analysis for target discovery in tuberculosis and provide a summary of potential TB drug targets identified by the individual methods. We also discuss current developments and future prospects for the application of systems biology in the field of TB target discovery.

The following is an excerpt from “Proteases in Mycobacterium Tuberculosis Pathogenesis: Potential as Drug Targets.” (See https://pubmed.ncbi.nlm.nih.gov/23642117/).

M. tuberculosis has a number of proteases with good potential as novel drug targets and developing drugs against these should result in agents that are effective against drug-resistant and drug-sensitive strains.

The following is an excerpt from “Serine Protease Activity Contributes to Control of Mycobacterium Tuberculosis in Hypoxic Lung Granulomas in Mice.” (See https://pubmed.ncbi.nlm.nih.gov/20679732/).

The hallmark of human Mycobacterium tuberculosis infection is the presence of lung granulomas . . . . These data suggest that serine protease activity acts as a protective mechanism within hypoxic regions of lung granulomas and present a potential new strategy for the treatment of tuberculosis.

The following is an excerpt from “Structure Determination of Mycobacterium tuberculosis Serine Protease Hip1 (Rv2224c).” (See https://www.ncbi.nlm.nih.gov/pmc/articles/PMC6033327/).

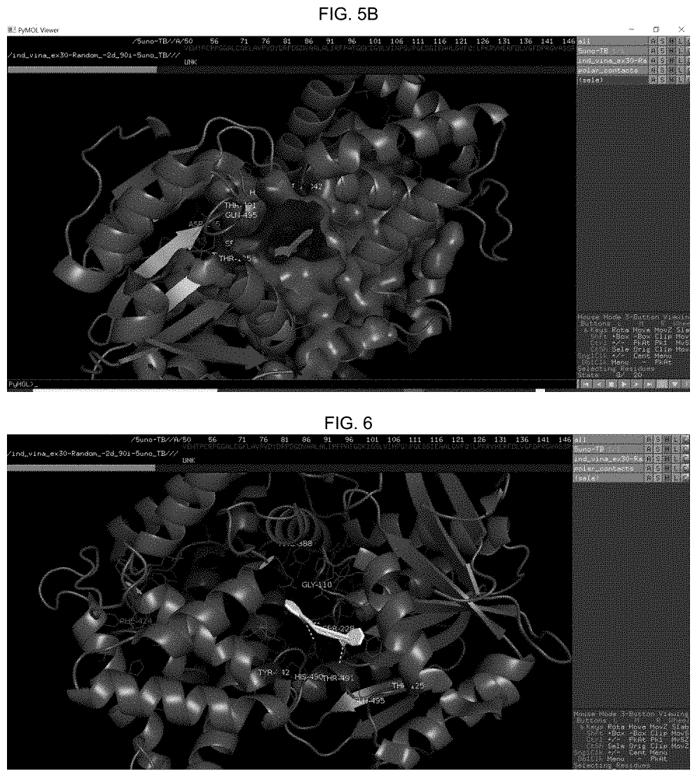

In the crystal structure, residues Ser228-Asp463-His490 are in close enough proximity to form a hydrogen bond network similar to those described for catalytic triads of serine proteases.²⁶ These residues are located in the α/β-domain within the cleft of the kidney-shaped protein and are at the center of the cavity (FIG. 5). The cavity is formed by parts of both the α/β- and α-domains and extends approximately 20 Å deep. The entrance to the cavity is lined with hydrophilic residues (Glu113, Glu117, Gln124, Thr125, Ser343, Asn345, Arg374, Asn382, and Gln495) with a hydrophobic pocket located at the upper part of the cavity (Met339, Tyr342, Leu346, Met 371, and Try 372). Close to the catalytic triad are three loops forming an active site pocket. One loop contains the catalytic residue Asp463, as well as residues Ala465 and Thr466. The other two loops are made up mainly of aliphatic residues; the first loop with residues Gly252 and Val254 lies at the bottom of the pocket. The second loop, on the opposite side of the pocket to the first loop, contains residues Gly109 and Gly110.

5UNO, Crystal Structure of Hip1 (Rv2224c). (See https://www.rcsb.org/structure/5UNO).

TABLE 8 [symbol]
log(ex30-Random)-2d_90i-5uno_TB - Notepad
File Edit Format View Help
WARNING: The search space volume >27000 Angstrom\hat{ }3 (See FAQ)
Detected 4 CPUs
Reading input . . . done.
Setting up the scoring function . . . done.
Analyzing the binding site . . . done.
Using random seed: 364216132
Performing search . . . done.
Refining results . . . done.

| | affinity | dist from best mode | |
|---|---|---|---|
| mode | (kcal/mol) | rmsd 1.b. | rmsd u.b. |
| 1 | −7.5 | 0.000 | 0.000 |
| 2 | −7.3 | 6.271 | 9.234 |
| 3 | −7.3 | 1.867 | 2.341 |
| 4 | −7.1 | 3.578 | 4.330 |
| 5 | −7.1 | 0.706 | 8.177 |
| 6 | −7.1 | 3.383 | 4.178 |
| 7 | −7.0 | 13.182 | 15.008 |
| 8 | −6.9 | 20.832 | 22.985 |
| 9 | −6.9 | 2.406 | 7.967 |
| 10 | −6.9 | 2.110 | 7.650 |
| 11 | −6.8 | 22.301 | 24.031 |
| 12 | −6.8 | 5.168 | 7.621 |
| 13 | −6.7 | 28.396 | 29.985 |
| 14 | −6.7 | 21.428 | 23.258 |
| 15 | −6.7 | 1.808 | 8.253 |
| 16 | −6.7 | 20.928 | 22.698 |
| 17 | −6.7 | 28.374 | 30.042 |
| 18 | −6.7 | 5.317 | 9.085 |
| 19 | −6.5 | 1.574 | 2.910 |
| 20 | −6.5 | 3.435 | 6.105 |

Writing output . . . done.

Referring to Table 8, a log file for 90I with different affinity values is included.

A binding with a protein-ligand complex and having the lowest energy, results in a better binding affinity. The benchmark is 5 kcal/mol or less is better, and an H bond of less than 3 Argon root-mean-square deviation (RMSD) to be an ideal distance from the residue atom that interacts to create an H bond.

However, even 5 Argon distance with more H bond may be sufficient for stability of the ligand when it interacts with the residues. 90I may have lots of H bonds with the residue of this protease.

90I may create more short distance H bonds with at least one residue indicating that it will interfere with Mycobacterium tuberculosis serine protease. As seen in the images below, 90I may form multiple H polar bonds.

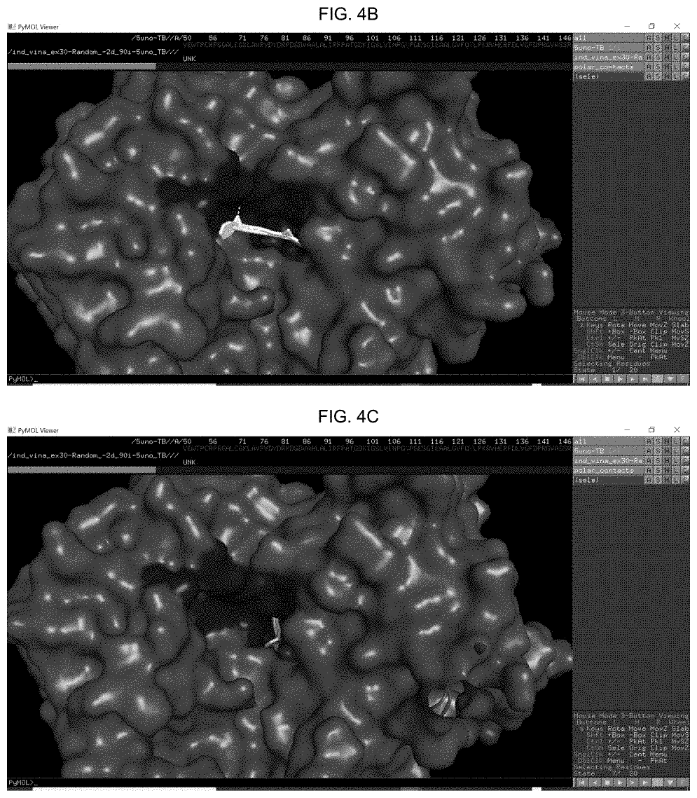

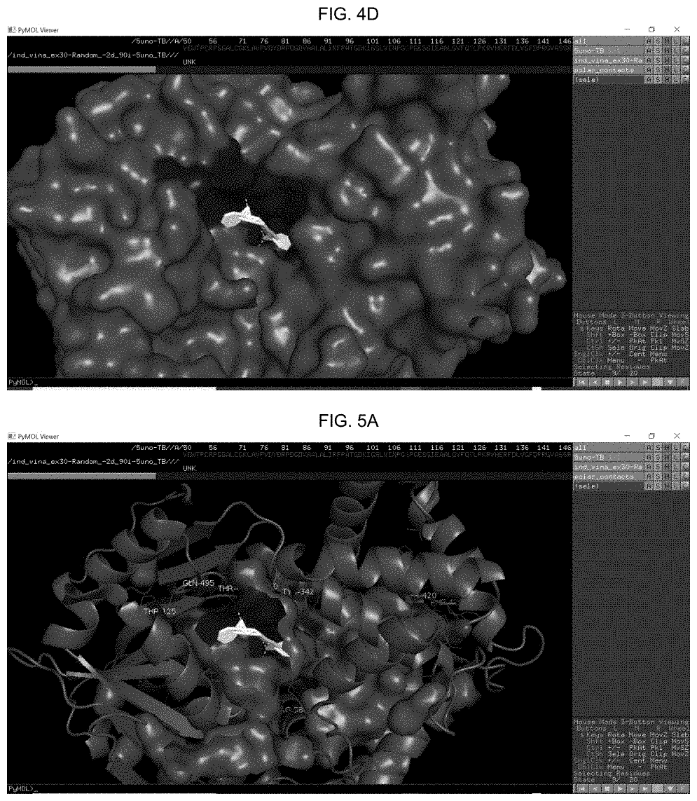

FIG. 4A illustrates a surface view of 90I interacting with a plurality of residues GLY 110 and THR 125, according to an exemplary embodiment of the general present inventive concept.

Referring to FIG. 4A, 90I is illustrated in white and the plurality of residues GLY 110 and THR 125 are in blue.

FIG. 4B illustrates the surface view of a first pose of 90I with an H bond interaction and an active site, according to an exemplary embodiment of the general present inventive concept.

Referring to FIG. 5B, 90I is illustrated in white and the active site is in blue.

FIG. 4C illustrates the surface view of 90I in white disposed deep inside the active site, according to an exemplary embodiment of the general present inventive concept.

FIG. 4D illustrates the surface view of 90I in white disposed deep inside the active site with three H bonds, according to an exemplary embodiment of the general present inventive concept.

FIG. 5A illustrates 90I disposed within a binding pocket interfering with Mycobacterium tuberculosis by interacting with active site residues, according to an exemplary embodiment of the general present inventive concept.

Referring to FIG. 5A, 90I is illustrated in white and the active site residues are in blue.

FIG. 5B illustrates 90I in white disposed within the binding pocket and interacting with a plurality of binding site residues in blue, according to an exemplary embodiment of the general present inventive concept.

Referring to FIG. 5B, 90I is illustrated in white and the plurality of binding site residues are in blue.

FIG. 6 illustrates 90I interacting with residues THR 491 and HIS 490 including two H bonds, according to an exemplary embodiment of the general present inventive concept.

Referring to FIG. 6, 90I is illustrated in white and the plurality of residues THR 491 and HIS 490 are in blue.

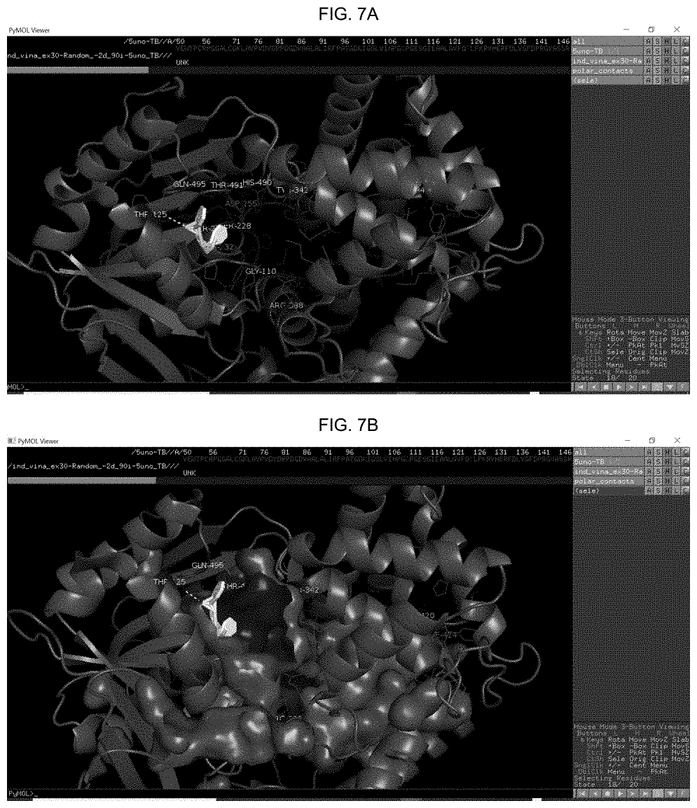

FIG. 7A illustrates 90I interacting with a residue THR 125 of the active site, according to an exemplary embodiment of the general present inventive concept.

Referring to FIG. 7A, 90I is illustrated in white and the residue THR 125 is in blue.

FIG. 7B illustrates a surface view of 90I disposed in a binding pocket interacting with the residue THR 125 of the active site, according to an exemplary embodiment of the general present inventive concept.

Referring to FIG. 7B, 90I is illustrated in white and the residue THR 125 is in blue.

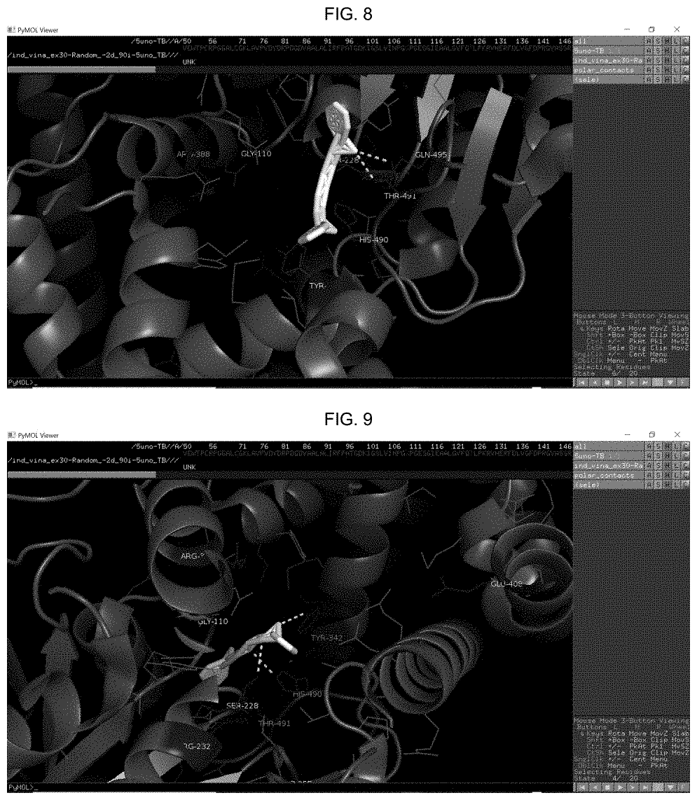

FIG. 8 illustrates another pose of 90I interacting with a residue GLN 495 in blue of the active site including two H bonds, according to an exemplary embodiment of the general present inventive concept.

Referring to FIG. 8, 90I is illustrated in white and the residue GLN 495 is in blue.

FIG. 9 illustrates 90I interacting with a plurality of residues including SER 228, TYR 342, and HIS 490 with two H bonds, according to an exemplary embodiment of the general present inventive concept.

Referring to FIG. 9, 90I is illustrated in white and the plurality of residues SER 228, TYR 342, and HIS 490 are in blue.

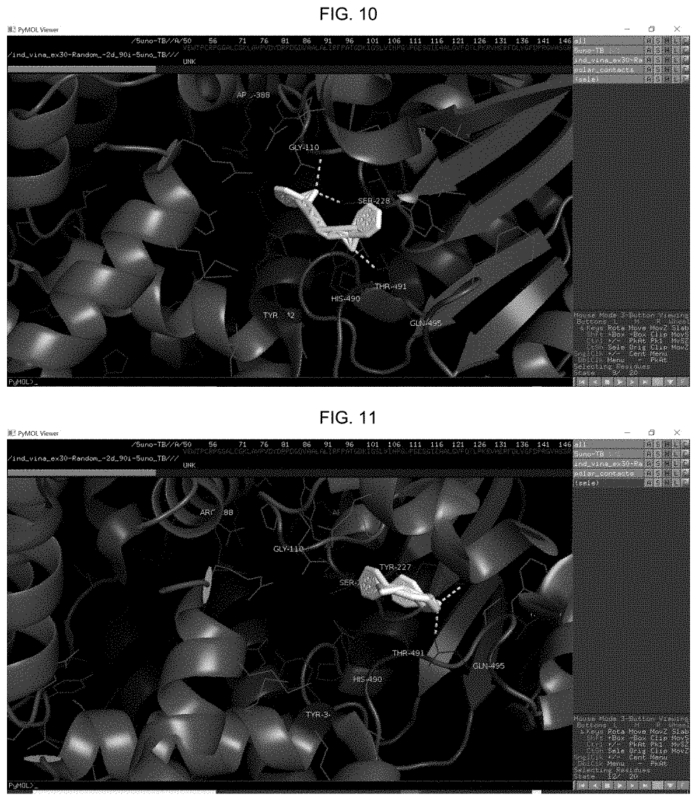

FIG. 10 illustrates 90I interacting with a plurality of residues GLY 110, SER 228, and THR 491 connected to HIS 490 including three H bonds, according to an exemplary embodiment of the general present inventive concept.

Referring to FIG. 10, 90I may have a strong connection to each of the three H bonds. More specifically, the 90I connection to each of the three H bonds is −6.9 kcal per mol and 2.406 Argon distance RMSD. Additionally, 90I is illustrated in white and the plurality of residues GLY 110, SER 228, and THR 491 are in blue.

FIG. 11 illustrates 90I interacting with a plurality of residues GLN 495 and TYR 227 connected to SER 228, according to an exemplary embodiment of the general present inventive concept.

Referring to FIG. 11, 90I is illustrated in white and the plurality of residues GLN 495, TYR 227, and SER 228 are in blue.

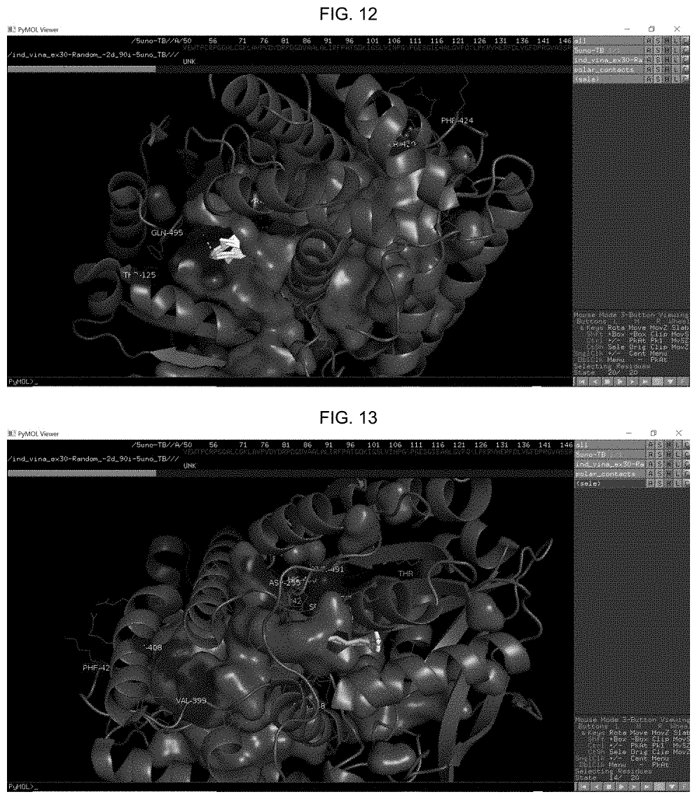

FIG. 12 illustrates 90I disposed within a binding pocket interacting with a residue GLN 495, according to an exemplary embodiment of the general present inventive concept.

Referring to FIG. 12, 90I is illustrated in white and the residue GLN 495 is in blue.

FIG. 13 illustrates 90I penetrating a binding pocket, according to an exemplary embodiment of the general present inventive concept.

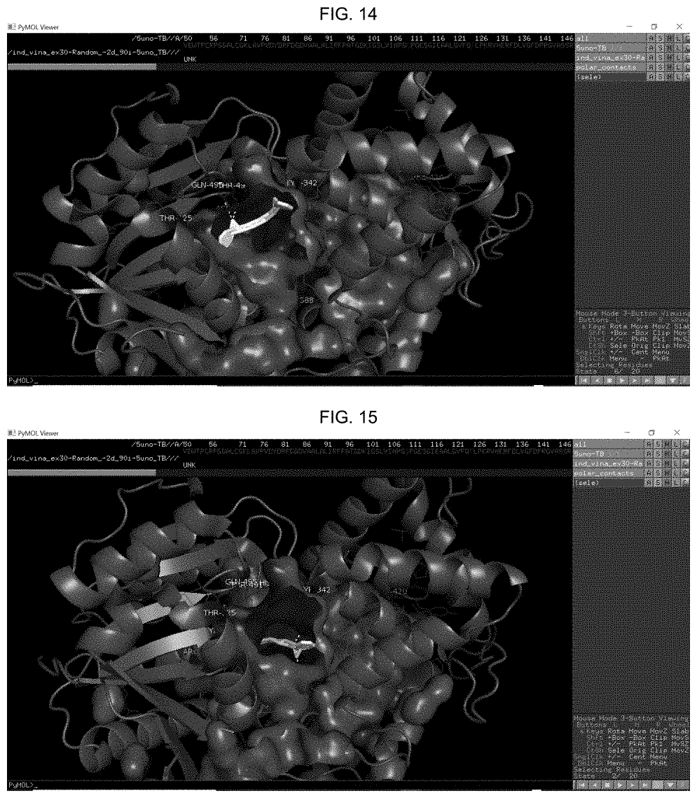

FIG. 14 illustrates 90I disposed within a binding pocket interfering with an active site residue GLN 495, according to an exemplary embodiment of the general present inventive concept.

Referring to FIG. 14, 90I is illustrated in white and the residue GLN 495 is in blue.

FIG. 15 illustrates 90I disposed deep within a binding pocket interacting with a plurality of active site residues, according to an exemplary embodiment of the general present inventive concept.

Referring to FIG. 15, 90I is illustrated in white and the plurality of active site residues are in blue.

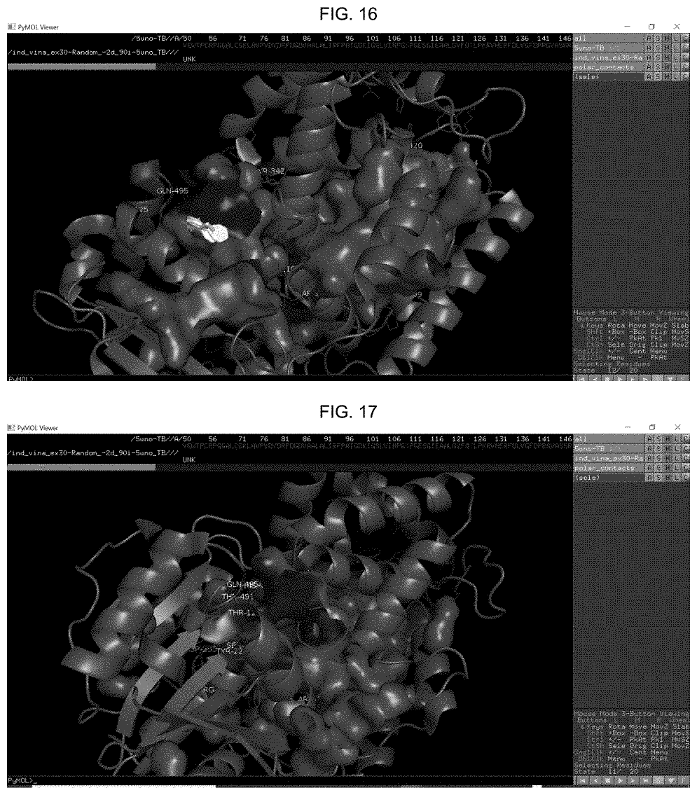

FIG. 16 illustrates a different pose of 90I disposed within a binding pocket, according to an exemplary embodiment of the general present inventive concept.

Referring to FIG. 16, 90I is illustrated in white.

FIG. 17 illustrates 90I disposed within a binding pocket, according to an exemplary embodiment of the general present inventive concept.

Referring to FIG. 17, 90I is buried under the binding pocket, which is located at a heart of a protein.

Proposed invention/s are all natural, low costing, and non-toxic treatments in targeting the most highly infectious parasitic disease which has crippled and burdened governments worldwide, especially third world countries. From the standpoint of the customer, most infectious cases ail individuals who cannot afford the current available treatments. Investing in these proposed inventions will alleviate the financial burden of the patients and decrease the need for treatment of side effects caused by the current available anti-TB drugs on the market.

Investigation of the presence of these antibodies in sera from healthy and ill subjects infected with M. tuberculosis in relation to the extent of the disease and the presence of IFN-gamma in sera by enzyme-linked-immunosorbent assay (ELISA). In order to investigate the presence of these antibodies at the site of infection we included 12 pleural fluids from tuberculosis patients and 9 pleural fluids from other origins. Toxicology testing will also be performed to assess appropriate dosing concentrations and volumes in Sprague-dawley rats (phase 1 and phase 2) and in dogs before submitting for review to continue on the clinical trials.

### REFERENCES

The following reference(s) may provide exemplary procedural and/or other details supplementary to those set forth herein, and are specifically incorporated herein by reference.
Freshney, R. I., 3^{rd} Edition (1994). Culture of Animal Cells: A Manual of Basic Technique. Wiley-Liss, Inc., New York, pp. 255-263.
Sher et al., 1992, Role of T cell derived cytokines in the down regulation of immune responses in parasitic and retroviral infection, Immunol, Rev. 127:183
Kornbluth et al., 1989, Interferon protects macrophages from productive infection by human immuno deficiency virus in vitro, J. Exp. Med. 169:137
K. Zaman. J Health Population Nutrition. Tuberculosis: A Global Health Problem. 2010 April; 28 (2):111-113
American Thoracic Society. “New clinical guideline for the treatment and prevention of drug resistant tuberculosis. ScienceDaily, 18 Nov. 2019. <www.sciencedaily.com/releases/2019/11/191118094105.htm>
Pathology of Tuberculosis, http://www.histopathology-india.net/Tuberculosis.htm.
Detection of Anti-Interferon-Gamma Autoantibodies in Subjects Infected by Mycobacterium Tuberculosis, https://pubmed.ncbi.nlm.nih.gov/9562113/.
In silico analyses for the discovery of tuberculosis drug targets, https://doi.org/10.1093/jac/dkt273.
Proteases in Mycobacterium Tuberculosis Pathogenesis: Potential as Drug Targets, https://pubmed.ncbi.nlm.nih.gov/23642117/.
Serine Protease Activity Contributes to Control of Mycobacterium Tuberculosis in Hypoxic Lung Granulomas in Mice, https://pubmed.ncbi.nlm.nih.gov/20679732/.
Structure Determination of Mycobacterium tuberculosis Serine Protease Hip1 (Rv2224c), https://www.ncbi.nlm.nih.gov/pmc/articles/PMC6033327/.
5UNO, Crystal Structure of Hip1 (Rv2224c), https://www.rcsb.org/structure/5UNO.

The present general inventive concept may include a method to diagnose bacterial infection, including phenotyping cell based resistant mycoplasm, amplification and identification of infection, and performing at least one assay on a cell culture.

The amplification may be a shell vial spin amplification method.

The identification may be a direct infected co-culture method, such that co-infection includes HIV.

The at least one assay may be based on ELISA.

The at least one assay may include at least one of ELISA IgG, IgM, and IgA.

The at least one assay may be based on ERBA LISA and another at least one assay is SEVA TB ELISA, which are performed simultaneously.

The bacterial infection may be caused by Mycobacterium tuberculosis.

The present general inventive concept may also include a method for the treatment of a bacterial disease, including administering to a subject in need thereof of an anti-pathogenic compound, such that the anti-pathogenic compound is derived from an herbal extract.

The herbal extract may be a glycol derivative.

The glycol derivative may be diethylene glycol dibenozate.

The bacterial disease may be caused by Mycobacterium tuberculosis.

The anti-pathogenic compound may boost an immune system of the subject.

The anti-pathogenic compound may boost the immune system by stimulating production of gamma interferon.

The anti-pathogenic compound may boost the immune system by inhibiting a protease enzyme of the bacterial disease.

The anti-pathogenic compound may boost the immune system by upregulating cellular genes.

Although a few embodiments of the present general inventive concept have been shown and described, it will be appreciated by those skilled in the art that changes may be made in these embodiments without departing from the principles and spirit of the general inventive concept, the scope of which is defined in the appended claims and their equivalents.

1. A method to diagnose bacterial infection, comprising:
phenotyping cell based resistant mycoplasm;
amplification and identification of infection; and
performing at least one assay on a cell culture.

2. The method of claim 1, wherein the amplification is a shell vial spin amplification method.

3. The method of claim 1, wherein the identification is a direct infected co-culture method, such that co-infection includes HIV.

4. The method of claim 1, wherein the at least one assay is based on ELISA.

5. The method of claim 1, wherein the at least one assay includes at least one of ELISA IgG, IgM, and IgA.

6. The method of claim 1, wherein the at least one assay is based on ERBA LISA and another at least one assay is SEVA TB ELISA, which are performed simultaneously.

7. The method of claim 1, wherein the bacterial infection is caused by Mycobacterium tuberculosis.

8. A method for the treatment of a bacterial disease, comprising:
administering to a subject in need thereof of an anti-pathogenic compound,
such that the anti-pathogenic compound is derived from an herbal extract.

9. The method of claim 7, wherein the herbal extract is a glycol derivative.

10. The method of claim 8, wherein the glycol derivative is diethylene glycol dibenzoate

11. The method of claim 7, wherein the bacterial disease is caused by Mycobacterium tuberculosis.

12. The method of claim 7, wherein the anti-pathogenic compound boosts an immune system of the subject.

13. The method of claim 11, wherein the anti-pathogenic compound boosts the immune system by stimulating production of gamma interferon.

14. The method of claim 11, wherein the anti-pathogenic compound boosts the immune system by inhibiting a protease enzyme of the bacterial disease.

15. The method of claim 11, wherein the anti-pathogenic compound boosts the immune system by upregulating cellular genes.
